(19) United States
(12) Patent Application Publication
(10) Pub. No.: US 2021/0052433 A1
(43) Pub. Date: Feb. 25, 2021

(54) BREASTFEEDING ASSISTANCE DEVICE

(71) Applicant: Allison M. CORDER, Wadsworth, IL (US)

(72) Inventor: Allison M. CORDER, Wadsworth, IL (US)

(21) Appl. No.: 16/888,712

(22) Filed: May 30, 2020

(51) Int. Cl. A61F 13/14

(52) U.S. Cl. CPC A61F 13/141

(57) ABSTRACT

The invention is directed to a device that simulates a mother's hold of her breast during breastfeeding. The device supports the breast in a “C”, “U”, or “V” hold by using support components on opposing surfaces of the breast.

### FIELD OF THE INVENTION

The invention generally relates to breastfeeding and specifically to an assistance device that supports a breast during breastfeeding.

### BACKGROUND OF THE INVENTION

Breastfeeding can be a beautiful and intimate way a mother can bond with her baby, and can contribute to a special and loving relationship.

There are many advantages to breastfeeding for both a baby and a mother. For the baby, breastfeeding is important to its physical and mental development. Breastmilk not only contains all the vitamins and nutrients a baby needs, particularly in the first six months of life, but also is packed with antibodies that protect the baby from illness. Today, many medical experts including the American Academy of Pediatrics (AAP) and the American College of Obstetricians and Gynecologists strongly recommend breastfeeding.

Numerous studies from around the world have shown that stomach viruses, respiratory illnesses, ear infections, and meningitis occur less often in breastfed babies and are less severe when they do happen. Other studies have shown that breastfeeding can protect a baby from developing allergies. And research confirms breastfeeding improves cognitive development.

Certain studies have shown that breastfeeding may also reduce a mother's risk of some types of cancer such as breast and ovarian cancer. Studies are finding new benefits of breastfeeding all the time. Like the published study showing that babies who are breastfed are less likely to have fevers after their immunizations than babies who are formula fed.

Breastfeeding can also reduce a new mother's stress level and risk of postpartum depression. It is known that breastfeeding triggers the release of the hormone oxytocin, which plays a role in behaviors including maternal-infant bonding and milk release. Also, breastfeeding burns extra calories that may help a new mother lose pregnancy weight faster.

During breastfeeding, oftentimes both hands of the mother are occupied—one hand to hold the baby in a breastfeeding position, such as behind the head, and the other hand to support the breast, such as to prevent the breast from covering the nose of the baby or weighing on the baby's lower jaw and chin, or to flatten the breast to encourage proper latch-on or to promote milk production.

A mother may use a particular hand position to assist with breastfeeding. The “C” hold, also known as the palmer grasp, is the most common hand position that mothers use to support the breast. Using the “C” hold as shown in FIG. 1A, the breast is placed in the palm of the hand. The thumb is positioned on the top of the breast, and the fingers are cupped around the bottom of the breast. The thumb and fingers are located behind the areola so as not to get in the way of the baby's mouth. The thumb and fingers can be gently squeezed together to flatten the areola and nipple like a sandwich, which may make it easier for some babies to latch-on.

One variation of the “C” hold is the “U” hold where support is provided under the breast. A hand cups the breast from underneath with the thumb going up one side of the breast and the fingers going up the other side.

Using the “V” hold as shown in FIG. 1B, the thumb and index finger are positioned on the top of the breast and the middle finger, ring finger and pinky finger are on the underside of the breast. The nipple and areola are placed between an index finger and the middle finger.

These “holds” may be troublesome for a mother to master or difficult to maintain during an entire breastfeeding session. Furthermore, in instances when both hands of the mother are occupied—one hand holding the baby and the other hand supporting the breast—it is difficult for a mother to perform additional activities, for example, interact with a mobile device, turn pages of a book, operate a television control device, write a note, or even eating or drinking.

What is needed is a device that simulates a mother's hold of her breast during breastfeeding. The present invention satisfies this need.

### SUMMARY OF THE INVENTION

The invention is directed to a device that simulates a mother's hold of her breast during breastfeeding. The device may assist with support of a mother's breast during breastfeeding, for example, to prevent the breast from covering the nose of the baby or weighing on the baby's lower jaw and chin, or to flatten the breast to encourage proper latch-on or to promote milk production.

The device supports the breast in a “C”, “U”, or “V” hold by using support components on opposing surfaces of the breast. Each support component simulates a thumb and/or finger(s) such that the breastfeeding assistance device simulates a mother's hand position during breastfeeding.

The device is easily operated and adjustable to various breast sizes. The mother may squeeze the support components together with one hand. Once the desired position is reached, the mother simply lets go of the device. Her hand is freed to perform additional activities. The support components are fixed, or locked, to remain in position so that the device supports the breast in a “C”, “U”, or “V” hold. After breastfeeding, the support components are released, or unlocked, so the device may be removed from the breast.

Each support component covers a portion of the breast surface and may be any configuration in terms of size, shape, length, thickness to support a breast. The support components may be shaped linear or curved such as to match the breast contour. In certain embodiments of the invention, the support components may further include a coating constructed of a material that overlays all or a portion of the support component. The coating may be constructed of any material such as fabric, microfiber, or foam that is comfortable for use against the skin. The coating may be quickly removable from the support component for cleaning.

The support components are separated by a gap that receives a portion of a breast. A connection component is connected to each of the support components. The connection component is configured to control movement of one or both of the support components to change a size of the gap. In one embodiment, the connection component is a mechanical mechanism configured to move the support components toward each other to decrease the gap and to move the support components apart from each other to increase the gap. In a preferred embodiment, the connection component is a ratchet-type mechanism that moves the support components toward each other while preventing movement in the reverse direction. Although the invention is described with respect to a ratchet-type mechanism, any equivalent mechanism is contemplated that can achieve the functions to move the support components toward each other and to move the support components apart from each other, and further, to lock and unlock the support components. Examples of equivalent designs may include, for example, cam-buckle mechanism, male-female mechanism, tongue-groove mechanism, one-way bearing mechanism, track-wheel mechanism, hinge-clip mechanism.

The components of the device may be constructed of any material such as those that are comfortable for use against the skin. The material may comprise a coefficient of friction to adhere to the skin and resist slipping. Materials that are also contemplated may be those that are dishwater safe. Or materials with temperature retentive or conductive properties. For example, the device may be warmed in a microwave or submerged in heated water. A warmed device may be desirable for comfort, or to facilitate the let-down reflex. A cooled device may be desirable to reduce swelling or soothe soreness. The material may also be non-absorbent or impermeable to fluids. Examples of materials include plastic, rubber, silicone, acrylic, nylon, rubber, or any combination.

The device is compact and lightweight making it easily portable and discreet.

The present invention and its attributes and advantages are further understood and appreciated with reference to the detailed description below of one contemplated embodiment, taken in conjunction with the accompanying drawings.

### DESCRIPTION OF THE DRAWINGS

The preferred embodiments of the invention are described in conjunction with the drawings provided to illustrate and not to the limit the invention, where like designations denote like elements.

FIG. 1A illustrates a “C” hold used during breastfeeding.

FIG. 1B illustrates a “V” hold used during breastfeeding.

FIG. 2A is a perspective view of a breastfeeding assistance device according to an embodiment of the invention.

FIG. 2B is another perspective view of the breastfeeding assistance device.

FIG. 3A is a perspective view of a first support component of the breastfeeding assistance device according to an embodiment of the invention.

FIG. 3B is a top view of the first support component.

FIG. 4A is a perspective view of a second support component of the breastfeeding assistance device according to an embodiment of the invention.

FIG. 4B is a top view of the second support component.

FIG. 5A is a perspective view of a body element of a connection component of the breastfeeding assistance device according to an embodiment of the invention.

FIG. 5B is a side view of the body element of the connection component.

FIG. 6 is a perspective view of a button element of the connection component of the breastfeeding assistance device according to an embodiment of the invention.

FIG. 7 is an exploded assembly view of the breastfeeding assistance device.

FIG. 8A is a perspective view of the breastfeeding assistance device.

FIG. 8B is a cross-section view of the breastfeeding assistance device in a locked state.

FIG. 9A is another perspective view of the breastfeeding assistance device.

FIG. 9B is a cross-section view of the breastfeeding assistance device in an unlocked state.

FIG. 10 is the breastfeeding assistance device as used in a “C” hold.

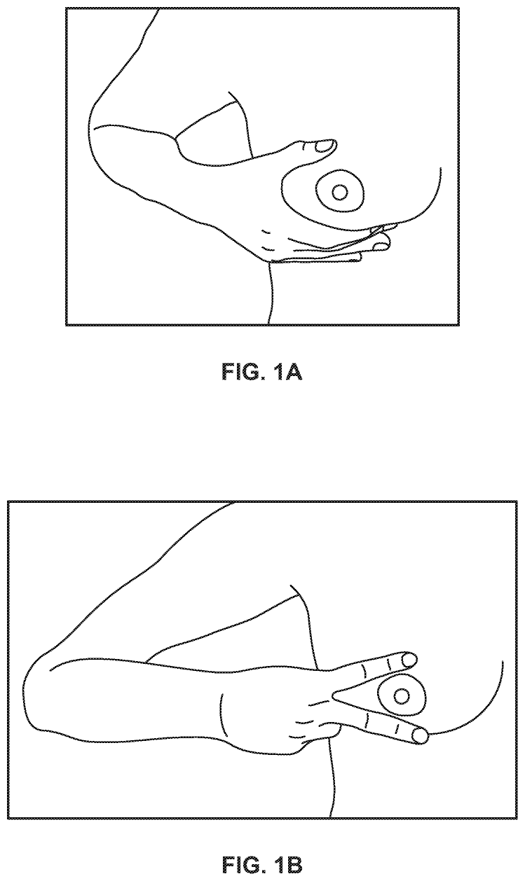

### DETAILED DESCRIPTION OF THE INVENTION

The invention is directed to a device that simulates a mother's hold of her breast during breastfeeding. The device supports the breast in a “C”, “U”, or “V” hold by using support components on opposing surfaces of the breast. A gap between the support components receives a portion of a breast. The support components are moveable with respect to each other such that the size of the gap varies.

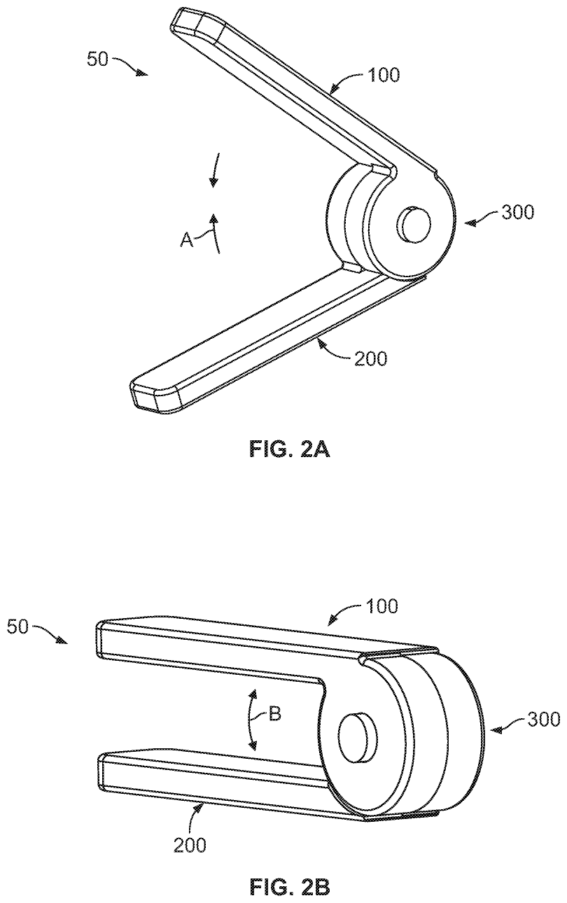

FIG. 2A and FIG. 2B illustrate a perspective view of an embodiment of the breastfeeding assistance device 50. The device 50 comprises two support components 100, 200 connected through a connection component 300. As shown by arrow “A” in FIG. 2A, the support components 100, 200 move toward each other to decrease the gap between the support components. As shown by arrow “B” in FIG. 2B, the support components 100, 200 move apart from each other to increase the gap between the support components.

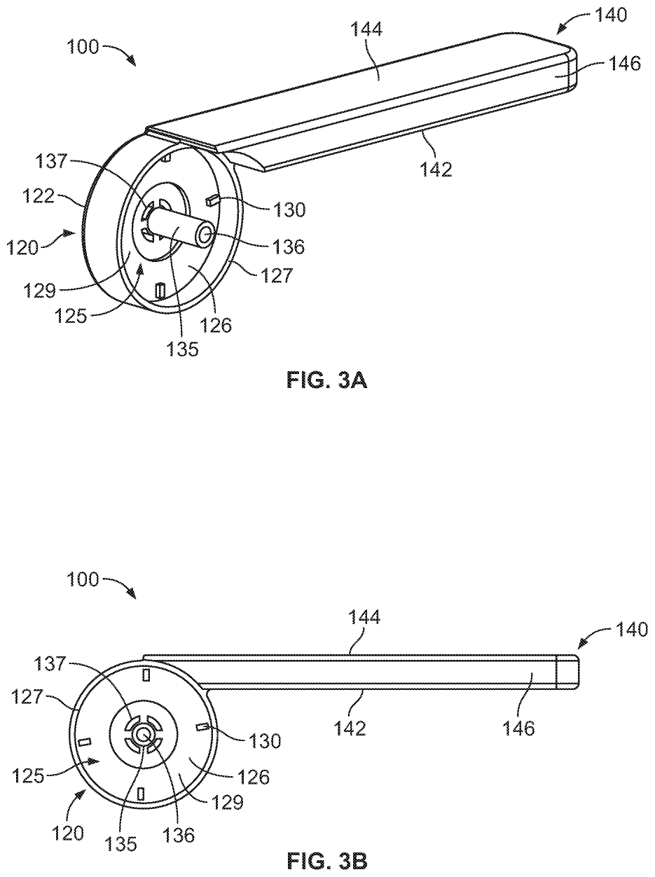

Each support component includes a hub portion and a projection portion. As shown in FIG. 3A and FIG. 3B, a first support component 100 includes a first projection portion 140 that extends from a first hub portion 120. The first projection portion 140 includes a lower surface 142 and an upper surface 144 with these surfaces 142, 144 connected by a border surface 146. Border surface 146 defines a thickness of the first projection portion 140.

The first hub portion 120 shown in FIG. 3A and FIG. 3B includes a cavity element 125. The cavity element 125 is defined by a base portion 126 surrounding by a boundary 127. The base portion 126 is defined by an exterior surface 122 and an interior surface 129. One or more pawl elements 130 are positioned on the interior surface 129 near the edge wall 127. The pawl elements 130 are shown as a rectangular cuboid, but any size and shape is contemplated. A spindle element 135 is centrally located within the cavity element 125. An opening 136 is located within the center of the spindle element 135. A plurality of aperture elements 137 surround the spindle element 135 with each aperture element 137 extending through both the exterior surface 122 and the interior surface 129.

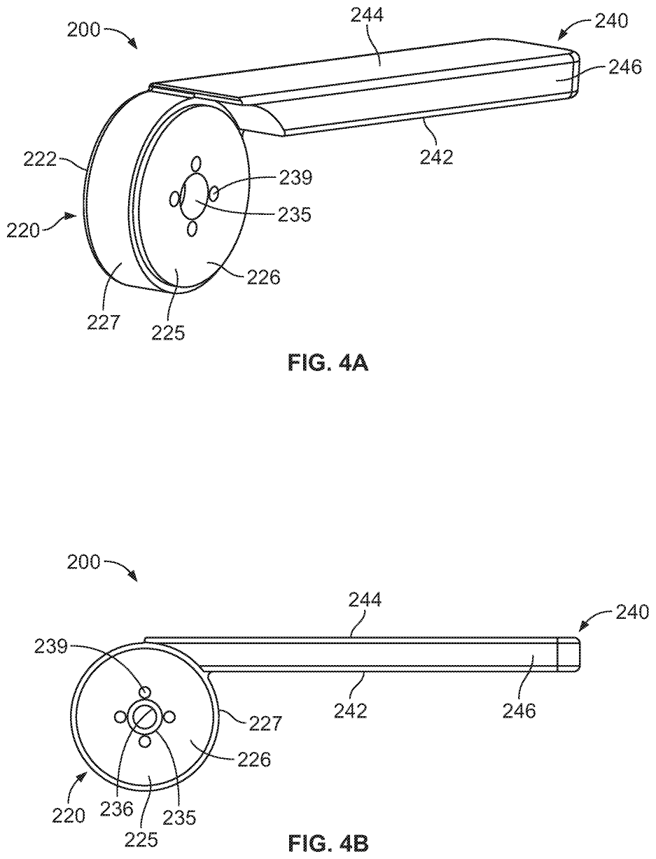

As shown in FIG. 4A and FIG. 4B, a second support component 200 has a second projection portion 240 that extends from a second hub portion 220. The second projection portion 240 includes an inner surface 242 and an outer surface 244. These surfaces 242, 244 are separated by a boundary surface 246 that defines a thickness of the second projection portion 240.

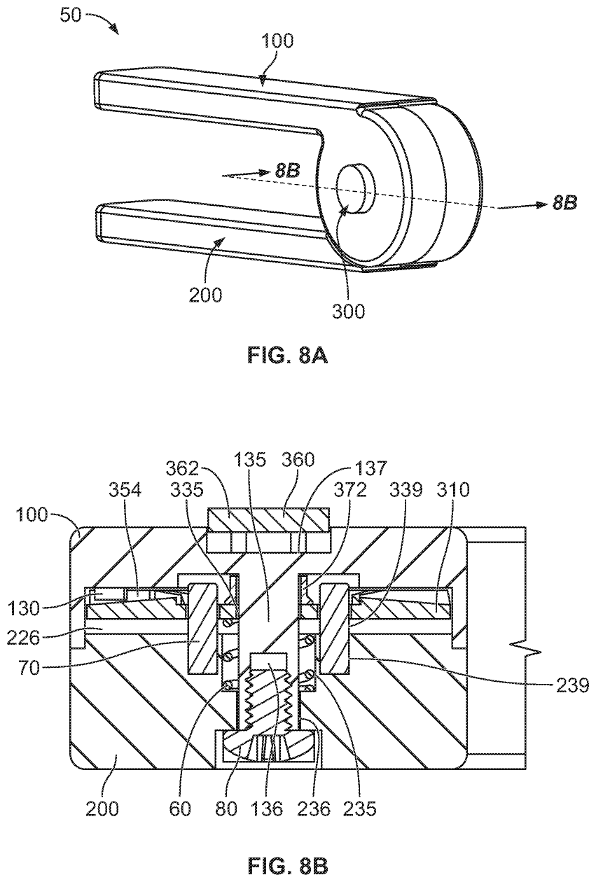

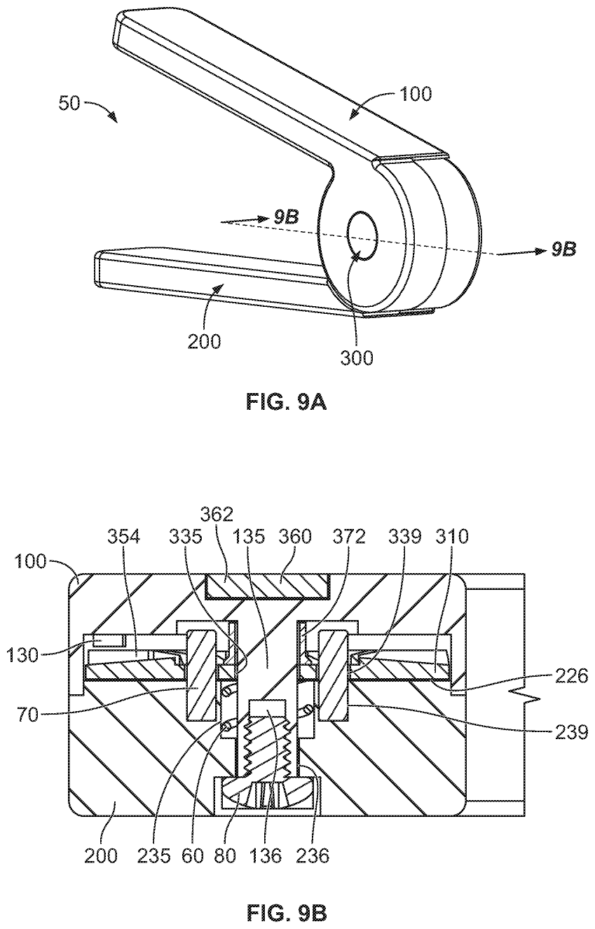

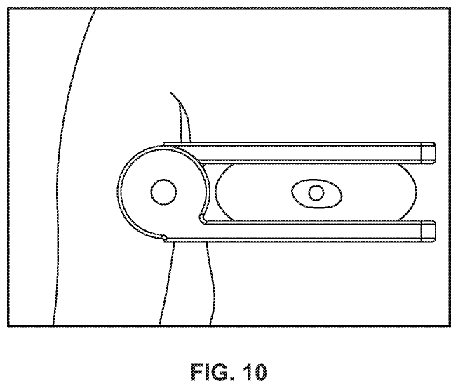

The second hub portion 220 shown in FIG. 4A and FIG. 4B includes a body element 225. The body element 225 is defined by an outside surface 222 and an inside surface 226 surrounded by a perimeter surface 227. A receptacle element 235 is centrally located in the body element 225. A plurality of pocket elements 239 are located near and surrounding the receptacle element 235. Each pocket element 239 extends through the inside surface 226 and terminates inside the body element 225. The receptacle element 235 further comprises a hollow chamber 236 (see also FIG. 8B and FIG. 9B).

The support components 100, 200 are sized and shaped to be comfortable against the skin while covering an area sufficient to support the breast. The support components are shown as a rectangular cuboid shape with linear surfaces and uniform thickness. However, it is contemplated that the support components may comprise any shape, any nonlinear surface, and any non-uniform thickness.

A connection component 300 connects the support components 100, 200. According to one embodiment, the connection component 300 is a ratchet-type mechanism that allows the support components 100, 200 to move toward each other while preventing movement in the reverse direction. In order to move in the reverse direction, the connection component is unlocked as discussed further below.

The connection component 300 includes a wheel element 310 and a button element 360. The wheel element 310 includes a top face 312 and a bottom face 314 separated by a border edge 316. Although the wheel element 310 is shown as a disc-shape, any size and shape is contemplated. The wheel element 310 includes a center opening 335 that extends through both the top face 312 and the bottom face 314. One or more holes 339 surround the center hole and also extend through both the top face 312 and bottom face 314. A plurality of tooth elements 350 are located on the top face 312. Although a plurality of tooth elements 350 are shown, it is contemplated that certain embodiments may only require one tooth element 350.

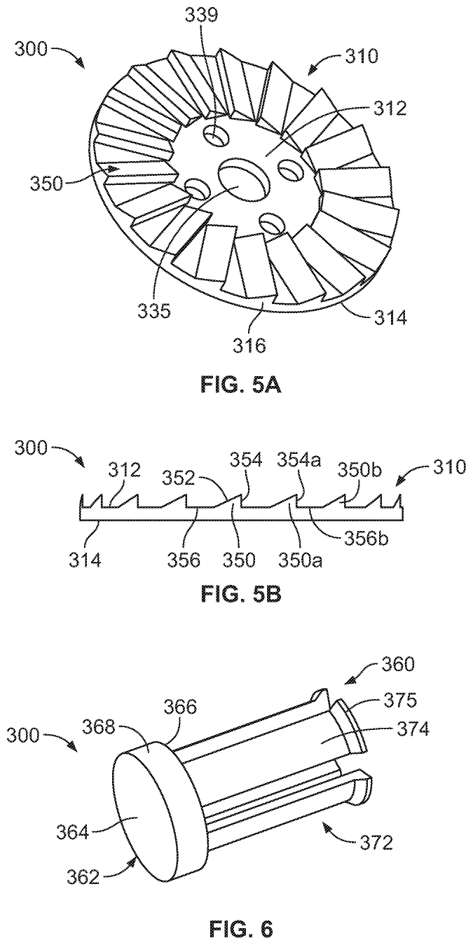

As shown more specifically in FIG. 5B, each tooth element 350 includes three surfaces: an angled surface 352 with a gentle slope connected between a vertical surface 354 with a much steeper slope and a horizontal surface 356. Two or more tooth elements 350 are connected at the point where the vertical surface 354a of one tooth element 350a meets the horizontal surface 356b of an adjacent tooth element 350b.

The connection component 300 may further comprise a button element 360 as shown in FIG. 6. The button element 360 is used to unlock the support components 100, 200 so that they can move apart from each other.

The button element 360 includes a head portion 362 and leg portion 372. The head portion 362 comprises a top surface 364 and a bottom surface 366. The surfaces 364, 366 are connected by a boundary surface 368. As shown in FIG. 6, the head portion 362 is a circular shape, but any size and shape is contemplated. The leg portion 372 extends from the head portion 362 and terminates at end 374. End 374 includes one or more pad elements 375.

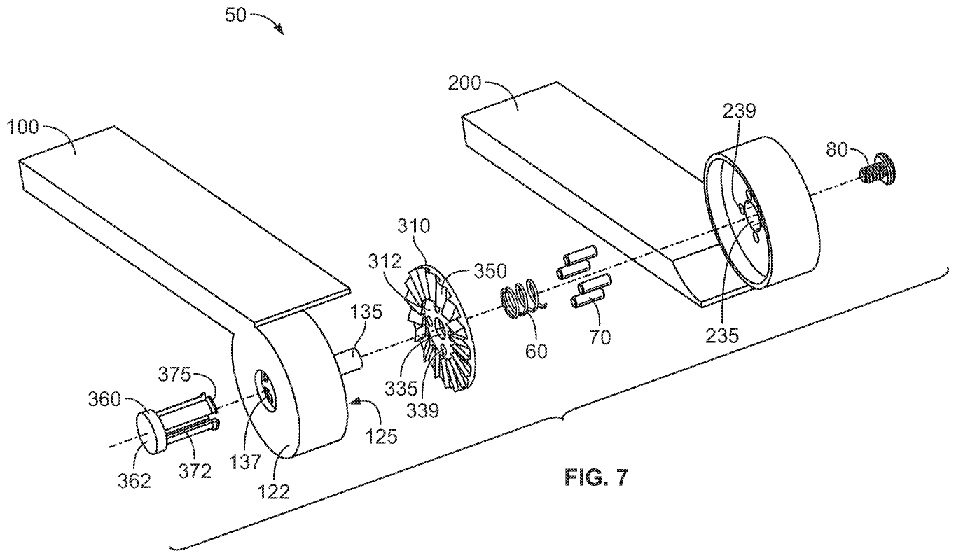

FIG. 7 illustrates an exploded assembly view of the breastfeeding assistance device 50 according to one embodiment of the invention.

The first support component 100 and the second support component 200 are assembled together. The button element 360 is assembled to the first support component 100 such that the head portion 362 protrudes from the exterior surface 122. Each leg portion 372 is routed through aperture element 137 such that pad element 375 is located within cavity element 125 and abuts the top face 312 of the wheel element 310. Pad element 375 prevents the button element 360 from separating from the device 50.

The wheel element 310 is assembled within the cavity element 125 with tooth elements 350 facing toward interior surface 129 of cavity element 125. Specifically, center opening 335 is positioned over spindle element 135 and spring 60 is coiled around spindle element 135. Pins 70 are used to connect wheel element 310 with support component 200. One end of pin 70 is positioned within pocket element 239 and the other end of pin 70 is positioned within hole 339 of wheel element 310. The spindle element 135 including spring 60 is positioned within receptacle element 235 such that a top portion of spindle element 135 is positioned within hollow chamber 236 (see FIG. 8B and FIG. 9B).

Center opening 335 and holes 339 of wheel element 310 are larger in diameter than the diameters of the spindle element 135 and posts 70 so that the wheel element 310 can move within the cavity element 125.

As shown, the first support component 100 and the second support component 200 are secured together by hardware 80, but any known assembly system or method is contemplated, e.g., snap-fit, male-female, threaded, or hardware connection. Hardware 80 is assembled within opening 136 of spindle element 135. FIG. 8B and FIG. 9B are cross-sectional views that also illustrate the assembly of device 50.

The device 50 is used to support a mother's breast during breastfeeding. The device 50 is positioned on a mother's breast so that the components 100, 200 are each positioned on opposing surfaces of a breast. The gap between the support components 100, 200 receives a portion of the breast. The mother squeezes the support components 100, 200 together with one hand to move them together, i.e., to decrease the size of the gap. Once the desired position is reached, the mother simply lets go of the device. Her hand is freed to perform additional activities. The support components are fixed, or locked, to remain in position so that the device supports the breast in a “C”, “U”, or “V” hold. After breastfeeding, the support components are released, or unlocked, so the device may be removed from the breast.

FIG. 8A is a perspective view of the breastfeeding assistance device and FIG. 8B is a cross-section view of the breastfeeding assistance device in a locked state.

Specifically, the wheel element 310 of the connection component 300 allows continuous rotary motion in only one direction while preventing motion in the opposite, or reverse, direction. As the support components 100, 200 are moved together, the wheel element 310 travels in an unrestricted (i.e., forward) direction. The pawl element 130 easily slides up and over the angled surface 352 of the tooth element 350, passes the tip of the tooth element 350, and down the vertical surface 354 toward the horizontal surface 356 of the adjacent tooth element.

When the wheel element 310 attempts to travel in the opposite (i.e., backward) direction, the pawl element 130 catches against the tooth element 350, specifically the steeply sloped vertical surface 354, locking it against the tooth element 350 and preventing any further motion in that direction.

FIG. 9A is another perspective view of the breastfeeding assistance device and FIG. 9B is a cross-section view of the breastfeeding assistance device in an unlocked state.

To unlock the support components 100, 200, and permit them to move apart from each other, button element 362 is pressed toward exterior surface 122. When the button element 362 is pressed, the pad element 375 of leg 372 pushes on the top face 312 of wheel element 310. This causes the bottom face 314 of wheel element 310 to move toward the inside surface 226 of support component 200. As a result, the pawl element 130 releases from the tooth element 350—i.e., a clearance is created such that the pawl element 130 does not catch against the tooth element 350—to allow the support components 100, 200 to move apart from each other, to reposition the device or to remove it from the breast.

For simplicity and clarity of illustration, where considered appropriate, reference numerals may be repeated among the Figures to indicate corresponding or analogous elements. In addition, numerous specific details are set forth in order to provide a thorough understanding of the embodiment described. However, it will be understood by those of ordinary skill in the art that the embodiment described may be practiced without these specific details. In other instances, well-known methods, procedures and components have not been described in detail so as not to obscure the embodiment described. Also, the description is not to be considered as limiting the scope of the embodiment described.

1. A breastfeeding assistance device comprising:
a first support component,
a second support component, the first support component and the second support component separated by a gap for receiving a portion of a breast, and
a connection component connected to each of the support components, the connection component configured to control movement of one or both of the support components to change a size of the gap.

2. The breastfeeding assistance device according to claim 1, wherein the connection component is a ratchet-type mechanism.

3. The breastfeeding assistance device according to claim 1, wherein the connection component comprises a wheel element.

4. The breastfeeding assistance device according to claim 3, wherein the wheel element comprises one or more tooth elements.

5. The breastfeeding assistance device according to claim 1, wherein the connection component comprises a button element.

6. The breastfeeding assistance device according to claim 1, further comprising a pawl element.

7. A breastfeeding assistance device for supporting a breast comprising:
a first support component configured to be positioned on a side of the breast,
a second support component configured to be positioned on an opposing side opposing side of the breast, wherein one or more of the first support component and the second support component are configured to move toward each other while preventing movement in the reverse direction.

8. The breastfeeding assistance device according to claim 7, wherein one or more of the first support component and the second support component are configured to move apart from each other.

9. A breastfeeding assistance device comprising:
a first support component including a first projection portion that extends from a first hub portion, the first hub portion including one or more pawl elements,
a second support component including a second projection portion that extends from a second hub portion,
a wheel element comprising one or more tooth elements, wherein the one or more pawl elements is configured to catch against the one or more tooth elements as the first support component and the second support component move toward each other.

10. The breastfeeding assistance device according to claim 9 further comprising a button element.

11. The breastfeeding assistance device according to claim 10, wherein the button element further comprises pad element, wherein the pad element is configured to push the wheel element such that the pawl element does not catch against the tooth element to allow the first support component and the second support component to move apart from each other.
